(19) United States
(12) Patent Application Publication
(10) Pub. No.: US 2024/0358076 A1
(43) Pub. Date: Oct. 31, 2024

(54) ATOMIZATION CORE, ATOMIZER, AND AEROSOL-FORMING APPARATUS

(71) Applicant: Jiangmen Smoore New Material Co., Ltd., Jiangmen (CN)

(72) Inventors: Yaqiao WANG, Jiangmen (CN); Gama YANG, Jiangmen (CN); Hui DAI, Jiangmen (CN)

(21) Appl. No.: 18/771,106

(22) Filed: Jul. 12, 2024

Related U.S. Application Data
(63) Continuation of application No. PCT/CN2023/071812, filed on Jan. 11, 2023.

(30) Foreign Application Priority Data
Jan. 13, 2022 (WO) PCT/CN2022/071861
Dec. 30, 2022 (CN) 202211738434.2

(51) Int. Cl. A24F 40/46; A24F 40/10; A24F 40/44; A24F 40/485; A61M 15/06; H05B 3/34

(52) U.S. Cl. CPC A24F 40/46; A24F 40/10; A24F 40/44; A24F 40/485; H05B 3/347; A61M 15/06

(57) ABSTRACT

An atomization core includes: a support member having a liquid guiding channel and a liquid guiding hole, the liquid guiding channel running through two ends of the support member in an axial direction of the support member, the liquid guiding hole running through a side wall of the liquid guiding channel in a radial direction of the support member; a liquid guiding core sleeved on an outer periphery of the support member, an aerosol-forming medium in the liquid guiding channel being guidable to the liquid guiding core through the liquid guiding hole; and a heating mesh arranged on an outer periphery of the liquid guiding core for heating and atomizing the aerosol-forming medium

### CROSS-REFERENCE TO PRIOR APPLICATION

This application is a continuation of International Patent Application No. PCT/CN2023/071812, filed on Jan. 11, 2023, which claims priority to International Patent Application No. PCT/CN2022/071861, filed on Jan. 13, 2022 and Chinese Patent Application No. 202211738434.2, filed on Dec. 30, 2022. The entire disclosure of each of the foregoing applications is hereby incorporated by reference herein.

### FIELD

This application relates to the field of atomization technologies, and in particular, to an atomization core, an atomizer, and an aerosol-forming apparatus.

### BACKGROUND

An atomization core of an aerosol-forming apparatus is configured to heat and atomize an aerosol-forming medium, to generate an aerosol. The aerosol is a colloidal dispersion system formed by dispersing and suspending small solid or liquid particles in a gaseous medium. Since the aerosol can be absorbed by the human body through the respiratory system, a novel alternative absorption manner is provided for users. The atomization core is generally formed by two parts: a cotton core and a heating element. In a production process, a forming process of the cotton core and a forming process of a heating wire are performed separately. After being produced, the cotton core and the heating wire are combined and formed by winding the heating wire around the cotton core, or wrapping the heating element with the cotton core.

In the related art, there are problems such as poor assembly consistency between the cotton core and the heating wire, uncontrollable spacings of the heating wire, and uneven spacings at a joint between the cotton core and a structural member, which leads to the problem that the process deviation of a turn spacing in the production process is prone to cause localized high temperature, resulting in poor taste consistency.

### SUMMARY

In an embodiment, the present invention provides an atomization core, comprising: a support member comprising a liquid guiding channel and a liquid guiding hole, the liquid guiding channel running through two ends of the support member in an axial direction of the support member, the liquid guiding hole running through a side wall of the liquid guiding channel in a radial direction of the support member; a liquid guiding core sleeved on an outer periphery of the support member, an aerosol-forming medium in the liquid guiding channel being guidable to the liquid guiding core through the liquid guiding hole; and a heating mesh arranged on an outer periphery of the liquid guiding core, the heating mesh being configured to heat and atomize the aerosol-forming medium

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an atomization core omitting a liquid guiding core according to an embodiment of this application;

FIG. 2 is a cross-sectional view of FIG. 1;

FIG. 3 is a cross-sectional view of an atomization core according to another embodiment of this application;

FIG. 4 is a schematic structural diagram of a support member according to an embodiment of this application;

FIG. 5 is a schematic structural diagram of a heating mesh according to an embodiment of this application; and

FIG. 6 is a schematic structural diagram of a connecting ring according to an embodiment of this application.

### DETAILED DESCRIPTION

In an embodiment, the present invention provides an atomization core, an atomizer, and an aerosol-forming apparatus, to resolve a problem of poor taste consistency due to localized high temperature caused by process deviation of a turn spacing of heating wires in a production process in the related art.

To achieve the above objective, an embodiment of this application provides an atomization core, including:
a support member including a liquid guiding channel and a liquid guiding hole, where the liquid guiding channel runs through the two ends of the support member in the axial direction of the support member, and the liquid guiding hole runs through the side wall of the liquid guiding channel in the radial direction of the support member;
a liquid guiding core, where the liquid guiding core is sleeved on the outer periphery of the support member, and an aerosol-forming medium in the liquid guiding channel can be guided to the liquid guiding core through the liquid guiding hole; and
a heating mesh, where the heating mesh is arranged on the outer periphery of the liquid guiding core, and the heating mesh is configured to heat and atomize the aerosol-forming medium.

In an embodiment, the heating mesh is a woven structure formed by weaving 4 to 64 heating wires.

In an embodiment, the diameter of the heating wire ranges from 0.01 mm to 0.5 mm.

In an embodiment, the material of the heating wire is iron chromium aluminum, nickel chromium, a titanium wire, or a nickel wire.

In an embodiment, the liquid guiding core is a cotton core.

In an embodiment, the cotton core includes 8 to 64 strands of yarn.

In an embodiment, the diameter of the yarn ranges from 0.05 mm to 2.0 mm.

In an embodiment, the fineness of the yarn ranges from 10 to 60.

In an embodiment, the porosity of the cotton core ranges from 0.45 to 0.99, and the permeability of the cotton core ranges from 1×10⁻¹¹ m² to 1×10⁻⁹ m².

In an embodiment, the thickness of the liquid guiding core ranges from 0.2 mm to 2 mm.

In an embodiment, the length of the support member ranges from 3 mm to 30 mm.

In an embodiment, the hole diameter of the liquid guiding hole is less than the inner diameter of the support member, and is greater than the effective capillary diameter of the liquid guiding core.

In an embodiment, the inner diameter of the support member ranges from 0.5 mm to 2.0 mm.

In an embodiment, the wall thickness of the support member ranges from 0.1 mm to 0.2 mm.

In an embodiment, the hole diameter of the liquid guiding hole ranges from 0.2 mm to 0.5 mm.

In an embodiment, the atomization core includes two connecting rings configured for electrical connection, and the two connecting rings are respectively sleeved on the two ends of the support member, and are electrically connected to the heating mesh.

In an embodiment, the two connecting rings are both sleeved on the heating mesh.

An embodiment of this application further provides an atomizer, including a housing and the above atomization core. A liquid storage cavity and an aerosol outlet channel are provided in the housing. The liquid storage cavity is configured to store an aerosol-forming medium. The aerosol outlet channel is configured for an aerosol to flow out of the atomizer. The aerosol-forming medium can flow into the liquid guiding channel through the liquid storage cavity.

An embodiment of this application further provides an aerosol-forming apparatus, including a power supply assembly and the above atomizer. The power supply assembly is electrically connected to the atomizer.

The embodiments of this application provide an atomization core, an atomizer, and an aerosol-forming apparatus. The atomization core includes a support member, a liquid guiding core, and a heating mesh. The support member includes a liquid guiding channel and a liquid guiding hole. The liquid guiding channel runs through the two ends of the support member in the axial direction of the support member. The liquid guiding hole runs through the side wall of the liquid guiding channel in the radial direction of the support member. An aerosol-forming medium in a liquid storage cavity of the atomizer can enter the liquid guiding channel through at least one end of the support member. The liquid guiding core is sleeved on the outer periphery of the support member, and covers the outer side of the liquid guiding hole. The aerosol-forming medium in the liquid guiding channel can be guided to the liquid guiding core through the liquid guiding hole. The heating mesh is arranged on the outer periphery of the liquid guiding core, and is configured to generate and atomize the aerosol-forming medium, to generate an aerosol for a user to inhale. For the atomization core in the embodiments of this application, the support member with specific rigidity is arranged, and the heating mesh of a mesh structure is woven and formed on the liquid guiding core on the outer periphery of the support member through weaving, so that heating meshes can be produced continuously, which is conducive to implementing automated assembly and can also improve assembly efficiency of the atomization core while ensuring that the heating mesh has good integrity and consistency. In this way, the temperature field distribution of the atomization core is more uniform, so that the aerosol has a good taste and good taste consistency, and the atomization area of the atomization core is also increased.

It should be noted that, without conflict, embodiments in this application and the technical characteristics in the embodiments can be combined with each other, and detailed description in specific embodiments should be understood as an explanation of the purpose of this application, and should not be regarded as an undue limitation of this application.

In the embodiments of this application, an “axial” orientation or positional relationship is an orientation or a positional relationship shown in FIG. 2. It should be understood that these orientation terms are only for the purpose of describing this application and simplifying the description, and do not indicate or imply that the referred apparatus or element needs to have a specific orientation, or be constructed and operated in a specific orientation. Therefore, the orientation terms cannot be understood as a limitation on this application. This application is described in further detail below with reference to the accompanying drawings and specific embodiments.

An embodiment of this application provides an aerosol-forming apparatus, including an atomizer and a power supply assembly. The power supply assembly is electrically connected to the atomizer. The atomizer is configured to store a liquid aerosol-forming medium, and heat and atomize the aerosol-forming medium under an action of electrical energy from the power supply assembly, to generate an aerosol for use by a user.

The aerosol-forming apparatus is configured to atomize the aerosol-forming medium to generate the aerosol for the user to inhale. The aerosol-forming medium includes, but is not limited to, pharmaceutical products, nicotine-containing materials, nicotine-free materials, or the like.

The aerosol-forming apparatus includes a liquid storage cavity configured to store the aerosol-forming medium. The aerosol-forming medium in the liquid storage cavity can flow to an atomization core 10 of the atomizer. The atomization core 10 is configured to heat and atomize the aerosol-forming medium to generate the aerosol. The generated aerosol can flow out of the aerosol-forming apparatus through an aerosol outlet channel of the atomizer for use.

It should be noted that a specific type of the aerosol-forming apparatus provided in the embodiments of this application is not limited. For example, the aerosol-forming apparatus may be a medical atomization device, an air humidifier, or an electronic cigarette.

An embodiment of this application provides an atomizer, including a housing and the atomization core 10.

A liquid storage cavity is provided in the housing. The liquid storage cavity is configured to store the aerosol-forming medium. The aerosol-forming medium can flow through the liquid storage cavity into a liquid guiding channel 11a of a support member 11 of the atomization core 10.

A liquid inlet channel and an aerosol outlet channel are provided in the housing. The aerosol outlet channel is configured for the aerosol to flow out of the atomizer. The aerosol-forming medium in the liquid storage cavity can flow into the liquid guiding channel 11a through the liquid inlet channel. In other words, the aerosol-forming medium in the liquid storage cavity of the atomizer can flow into the liquid guiding channel 11a through the liquid inlet channel. A heating mesh 13 heats and atomizes the aerosol-forming medium to form the aerosol, and the aerosol flows through the aerosol outlet channel out of the atomizer for use by the user.

It should be noted that a specific manner of using the atomizer is not limited herein. For example, the user may inhale the aerosol through the housing, or may inhale the aerosol through an additional mouthpiece cooperating with the housing.

It should be noted that a specific shape of the housing is not limited herein. The shape of the housing includes, but is not limited to, a hollow cylindrical shape, a hollow elliptical cylindrical shape, or the cross section shape of the housing is a rounded polygon, for example, a rounded triangle.

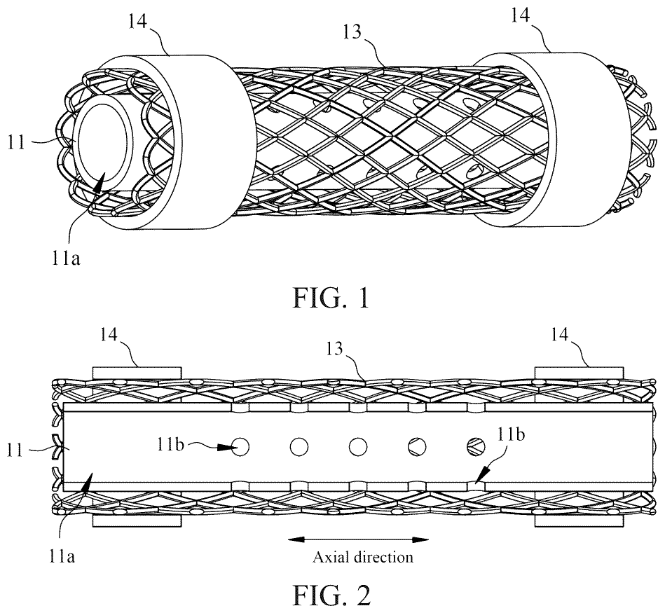

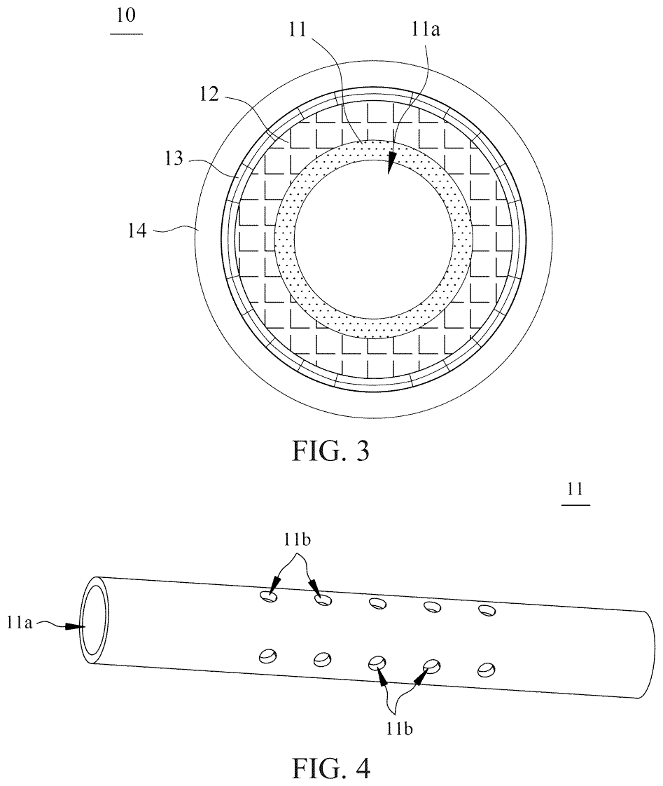

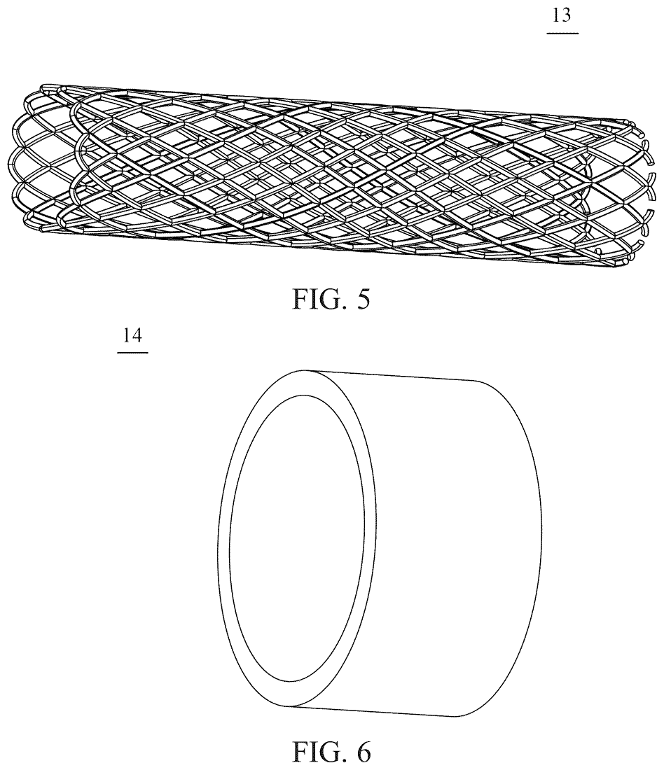

An embodiment of this application provides an atomization core, referring to FIG. 1 to FIG. 5, including the support member 11, the liquid guiding core 12, and the heating mesh 13.

The support member 11 includes a liquid guiding channel 11a and a liquid guiding hole 11b. The liquid guiding channel 11a runs through the two ends of the support member 11 in the axial direction of the support member 11. The liquid guiding hole 11b runs through the side wall of the liquid guiding channel 11a in the radial direction of the support member 11. The aerosol-forming medium in the liquid storage cavity can enter the liquid guiding channel 11a through at least one end of the support member 11. The liquid guiding core 12 is sleeved on the outer periphery of the support member 11, and covers the outer side of the liquid guiding hole 11b. The aerosol-forming medium in the liquid guiding channel 11a can be guided to the liquid guiding core 12 through the liquid guiding hole 11b. The heating mesh 13 is arranged on the outer periphery of the liquid guiding core 12. The heating mesh 13 is configured to heat and atomize the aerosol-forming medium to generate the aerosol for the user to inhale.

Certainly, the aerosol-forming medium in the liquid storage cavity may alternatively be not guided to the liquid guiding core 12 through the liquid guiding channel 11a and the liquid guiding hole 11b, but be directly absorbed by the part of the liquid guiding core 12 that extends into the liquid storage cavity or in communication with the liquid storage cavity.

It should be noted that one end of the liquid guiding channel 11a may be in communication with the liquid storage cavity, or both ends of the liquid guiding channel 11a may be in communication with the liquid storage cavity. An example in which the both ends of the liquid guiding channel 11a are in communication with the liquid storage cavity is used in this embodiment of this application for description. In this way, an amount of the aerosol-forming medium entering the atomization core 10 is increased, and the aerosol-forming medium can be guided to the liquid guiding core 12 uniformly through the liquid guiding channel 11a, thereby improving an atomization effect.

For example, a woven mesh (mesh)-shaped heating mesh 13 is produced by a weaving machine, and has good consistency and integrity. Density and resistance of the heating mesh 13 can be controlled through adjusting parameters and a weaving speed of the weaving machine.

It should be noted that the weaving texture of the heating mesh 13 may be a twill texture, a cross texture, or a herringbone texture, which may be specifically set according to actual needs.

It should be noted that the support member 11 as an example in the embodiments of this application may be a tubular member for accommodating the aerosol-forming medium. The tubular member is in a shape similar to a cylinder, but is not intended to limit the shape of the support member 11 in the embodiments of this application to being similar to a cylinder. The support member 11 in the embodiments of this application may alternatively be in a shape similar to a triangle or an elliptical cylinder, or another shape.

For example, referring to FIG. 1 to FIG. 4, the support member 11 is a hollow tube with the two ends penetrated, the tube cavity of the hollow tube is constructed into the liquid guiding channel 11a, and the liquid guiding hole 11b runs through the side wall of the hollow tube in the radial direction of the hollow tube.

It should be noted that a specific shape of the liquid guiding hole 11b, which is not limited herein, includes, but is not limited to, a circular hole, an elliptical hole, an elongated hole, a square hole, or the like.

It should be noted that a specific shape of the liquid guiding channel 11a is not limited herein. The cross-sectional shape of the liquid guiding channel 11a includes, but is not limited to, a circle, an ellipse, or a rounded polygon, for example, a rounded triangle. For example, in an embodiment, referring to FIG. 1 to FIG. 4, the cross-sectional shape of the liquid guiding channel 11a is a circle, which is conducive to smoothness of flow of the aerosol-forming medium in the liquid guiding channel 11a. The cross-sectional shape of the liquid guiding channel 11a is the cross-sectional shape of the liquid guiding channel 11a along a plane perpendicular to the axial direction of the support member 11.

It should be noted that a specific material of the support member 11 is not limited herein. For example, the material of the support member 11 is, for example, stainless steel, dense ceramic, or porous ceramic. In this way, the structural strength of the support member 11 is ensured, so that other components such as the liquid guiding core 12 and the heating mesh 13 of the atomization core 10 can be effectively supported.

In some embodiments, the support member 11 is made of glass. The glass is, specifically, any one of borosilicate glass, quartz glass, or photosensitive lithium aluminosilicate glass. In some embodiments, the support member 11 is made of stainless steel. The stainless steel has advantages such as high strength and good machinability, so that when several liquid guiding holes are provided on the side wall of the support member 11, stability of the support member can be ensured. Specifically, the support member may be made of SUS304 stainless steel, which has characteristics of low toxicity and high thermal conductivity, is suitable for contact with an e-liquid in the electronic atomization apparatus, and has high safety performance, and fluidity of the e-liquid is excellent. In some other embodiments, the support member 11 may be made of materials with specific mechanical strength such as ceramic, other metals, or rigid plastic.

In the related art, the atomization core is generally formed by two parts: a cotton core and a heating element. In a production process, a forming process of the cotton core and a forming process of a heating wire are performed separately. After the cotton core and the heating wire are produced, the heating wire winds around cotton, or the cotton core wraps the heating wire, to perform combined forming. In an existing atomization core in which a heating wire winds around a cotton core, in a production process, winding of the cotton core and the heating wire needs to be performed separately. After being formed, the cotton core needs to be cut and wound around the heating wire individually and manually, so that assembly consistency is poor, and efficiency is low. Moreover, a wound heating element has a small atomization area, temperature distribution is not uniform, and taste consistency is easily affected by a turn spacing. An existing atomization core in which a cotton core wraps a heating element has good taste, but is produced by stamping or etching processes. In a production process, individual sheet heating elements are first obtained, and curled manually and wrapped in the cotton core one by one. A process of manual curling and wrapping has low efficiency and poor consistency, the adopted etching process has high costs, and automated assembly has a complex process.

However, the atomization core 10 provided in the embodiments of this application includes the support member 11, the liquid guiding core 12, and the heating mesh 13. The support member 11 includes the liquid guiding channel 11a and the liquid guiding hole 11b. The liquid guiding channel 11a runs through the two ends of the support member 11 in the axial direction of the support member 11. The liquid guiding hole 11b runs through the side wall of the liquid guiding channel 11a in the radial direction of the support member 11. The aerosol-forming medium in the liquid storage cavity of the atomizer can enter the liquid guiding channel 11a through at least one end of the support member 11. The liquid guiding core 12 is sleeved on the outer periphery of the support member 11, and covers the outer side of the liquid guiding hole 11b. The aerosol-forming medium in the liquid guiding channel 11a can be guided to the liquid guiding core 12 through the liquid guiding hole 11b. The heating mesh 13 is arranged on the outer periphery of the liquid guiding core 12, and is configured to generate and atomize the aerosol-forming medium, to generate the aerosol for the user to inhale. In the atomization core 10 in the embodiments of this application, the support member 11 with specific rigidity is arranged, and the heating mesh 13 of a mesh structure is woven and formed on the liquid guiding core 12 on the outer periphery of the support member 11 through weaving, so that the heating meshes 13 can be produced continuously. This is conducive to implement automated assembly, can further improve assembly efficiency of the atomization core 10, and can ensure that the heating mesh 13 has good integrity and consistency. In this way, temperature field distribution of the atomization core 10 is more uniform, so that the aerosol has good taste, taste consistency is good, and an atomization area of the atomization core 10 is further increased.

The atomization core 10 provided in the embodiments of this application includes the support member 11, the liquid guiding core 12, and the heating mesh 13. The heating mesh 13 of the mesh structure is woven and formed on the liquid guiding core 12 on the outer periphery of the support member 11 through weaving, resolving the problem of manual winding the heating wire around the liquid guiding core 12, and resolving the problems of high manufacturing costs of the liquid guiding core 12 wrapping the heating element and complex automated assembly.

In an embodiment, the heating mesh 13 is a woven structure formed by weaving 4 to 64 heating wires. In other words, the 4 to 64 heating wires are woven to form the heating mesh 13 by using a weaving machine, so that excellent integrity and consistency of the heating mesh 13 can be ensured, and the density and the resistance of the heating mesh 13 can be controlled by adjusting the parameters and the weaving speed of the weaving machine. The heating mesh 13 is formed by weaving 4 to 64 heating wires, the temperature field distribution and the atomization area of the atomization core 10 can be controlled by controlling a number of heating wires, so that heating efficiency of the atomization core 10 is controlled. That is, the number of the heating wires can be controlled to adapt to aerosol-forming apparatuses of different types and demands.

In an embodiment, the diameter of the heating wire ranges from 0.01 mm to 0.5 mm. For example, the diameter of the heating wire is, for example, 0.01 mm, 0.05 mm, 0.08 mm, 0.1 mm, 0.15 mm, 0.2 mm, 0.25 mm, 0.3 mm, 0.35 mm, 0.38 mm, 0.4 mm, 0.45 mm, or 0.5 mm. It may be understood that if the diameter of the heating wire is too small, the structural strength of the heating wire is low, the heating wire is easily broken in a weaving process, and the structural strength of the heating mesh 13 formed by weaving is low, thereby reducing a service life of the atomization core 10. If the diameter of the heating wire is too large, the woven heating mesh 13 is easy to be excessively thick, which is not conducive to the flow of the aerosol-forming medium and affects an atomization amount of the aerosol-forming apparatus. The diameter of the heating wire is limited between 0.01 mm and 0.5 mm, so that it is ensured that the heating mesh 13 has specific structural strength, and the atomization amount of the aerosol-forming apparatus is improved. In addition, the diameter of the heating wire is limited between 0.01 mm and 0.5 mm, so that the heating efficiency of the atomization core 10 is ensured.

It should be noted that the material of the heating wire is not limited herein. For example, in some embodiments, the material of the heating wire is a metal or an alloy such as iron chromium aluminum, nickel chromium, a titanium wire, or a nickel wire.

In an embodiment, the liquid guiding core 12 is a cotton core, and a specific material of the cotton core is not limited. For example, the material of the cotton core may be natural organic cotton or organically synthesized polymer porous foam cotton.

The cotton core is made of a cotton fiber material. The cotton core is formed by, for example, a cotton thread or a cotton yarn, and can stably store the part of the aerosol-forming medium, and quickly guide the aerosol-forming medium in the liquid guiding channel 11a to the heating mesh 13. The heating mesh 13 heats the aerosol-forming medium on a cotton layer in an energized state, to form the aerosol.

The cotton core may be bulk cotton or woven cotton, and the materials include, but are not limited to, cotton yarn, linen, viscose, polyester, polyimide, or other fiber materials.

In an embodiment, the cotton core includes 8 to 64 strands of yarn. In other words, the cotton thread or the cotton yarn is formed by weaving 8 to 64 strands of yarn, so that it can be ensured that the woven cotton core has the set thickness and flowability, causing the cotton core to stably store the part of the aerosol-forming medium, and quickly guide the aerosol-forming medium in the liquid guiding channel 11a to the heating mesh 13.

In an embodiment, the diameter of the yarn ranges from 0.05 mm to 2.0 mm. For example, the diameter of the yarn is, for example, 0.05 mm, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm. It may be understood that if the diameter of the yarn is too small, the woven cotton core has low structural strength, and the service life of the atomization core 10 is reduced. If the diameter of the yarn is too large, the woven cotton core is easy to be excessively thick, which is not conducive to the flow of the aerosol-forming medium and affects an atomization amount of the aerosol-forming apparatus. The diameter of the yarn is limited between 0.05 mm and 0.2 mm, so that it can be ensured that the cotton core has specific structural strength, and the atomization amount of the aerosol-forming apparatus is improved.

In an embodiment, the fineness of the yarn ranges from 10 to 60. It may be understood that a value range of the fineness of the yarn is further limited, so that it can be ensured that the cotton core has specific structural strength, and the atomization amount of the aerosol-forming apparatus is improved.

In an embodiment, the thickness of the cotton core ranges from 0.2 mm to 2.0 mm. When the thickness of the cotton core is less than 0.2 mm, the thickness of the cotton core is too small, which is prone to leakage. When the thickness of the cotton core is greater than 2.0 mm, a flow path of the liquid in the cotton core is excessively long. The thickness of the cotton core is set between 0.2 mm and 2.0 mm, so that the flow path of the liquid in a porous structure in the cotton core can be reduced, and leakage does not occur.

In an embodiment, the porosity of the cotton core ranges from 0.45 to 0.99, and the permeability of the cotton core ranges from 1×10⁻¹¹ m² to 1×10⁻⁹ m². The permeability is a capability of allowing a fluid to pass under a specific pressure difference, and is a parameter indicating a liquid guide capability of the cotton core. When the permeability of the cotton core is less than 1×10⁻¹¹ m², a liquid supply amount is affected, and a vapor amount is reduced. When the permeability of the cotton core is greater than 1×10⁻⁹ m², the leakage may be caused. When the porosity of the cotton core is less than 0.45, the liquid supply amount is affected, and the vapor amount is reduced. When the porosity of the cotton core is greater than 0.99, the structural strength of the cotton core is affected. In this way, it is ensured that the cotton core has a specific liquid supply speed, and the leakage can be avoided.

In an embodiment, the hole diameter of the liquid guiding hole 11b is less than the inner diameter of the support member 11, and is greater than the effective capillary diameter of the liquid guiding core 12. It may be understood that the cotton core has a plurality of capillary holes. The plurality of capillary holes are in communication with the liquid guiding hole 11b. The hole diameter of the liquid guiding hole 11b is less than the inner diameter of the support member 11, and the hole diameter of the liquid guiding hole 11b is greater than the diameter of the capillary hole, so that effective flow of the aerosol-forming medium can be ensured, the cotton core can quickly guide the aerosol-forming medium in the liquid guiding channel 11a to the heating mesh 13 under a capillary force of the capillary holes, and the heating mesh 13 heats the aerosol-forming medium on the cotton layer in the energized state, to generate the aerosol.

In an embodiment, the inner diameter of the support member 11 ranges from 0.5 mm to 2.0 mm. For example, the diameter of the yarn is, for example, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm. In other words, a value range of the inner diameter of the support member 11 is limited, that is, a value range of the size of the liquid guiding channel 11a is limited. It may be understood that if the inner diameter of the support member 11 is too small, that is, the size of the liquid guiding channel 11a is too small, so that an amount of the aerosol-forming medium flowing in a unit volume is small, and a liquid supply speed of the atomization core 10 is reduced. If the inner diameter of the support member 11 is too large, so that the volume of the atomization core 10 is large, an inner space of the aerosol-forming apparatus is occupied, and the volume of the aerosol-forming apparatus is large, which is not conducive to the appearance of the product. The inner diameter of the support member 11 is limited between 0.5 mm and 2.0 mm, so that it is ensured that the atomization core 10 has a specific liquid supply speed, and the beauty of the product is improved.

In an embodiment, the wall thickness of the support member 11 ranges from 0.1 mm to 0.2 mm. For example, the wall thickness of the support member 11 is, for example, 0.1 mm, 0.12 mm, 0.14 mm, 0.15 mm, 0.16 mm, 0.18 mm, or 0.2 mm. It may be understood that in a case that the strength and safety are ensured, the wall thickness of the support member 11 can be reduced as much as possible. In this way, mass of the support member 11 is reduced, so that heat capacity consumption of the support member 11 is reduced, and the heating efficiency of the atomization core 10 is improved. In addition, in a case of the same outer diameter, the inner diameter of the support member 11 can be larger, and macroscopic flow resistance in the liquid guiding channel 11a can be smaller. In addition, the wall thickness of the support member 11 is reduced, so that a path of the aerosol-forming medium flowing from the liquid guiding channel 11a to the liquid guiding core 12 is reduced, and flow resistance of the aerosol-forming medium is further reduced, thereby improving a liquid supply capability and an atomization effect of the atomization core 10.

In some embodiments, the length of the support member 11 ranges from 3 mm to 30 mm. It may be understood that the axial length of the support member 11 is larger, the cotton core is longer correspondingly, coverage of the heating mesh 13 is larger, and a liquid supply area of the support member 11 is larger. In this way, if the axial length of the support member 11 is too small, the liquid supply capability and the atomization efficiency of the atomization core 10 are reduced. If the axial length of the support member 11 is too large, the volume of the atomization core 10 is large, so that the inner space of the atomizer is occupied, and the volume of the atomizer is large, which is not conducive to the beauty of the product. The axial length of the support member 11 is limited between 3 mm and 30 mm, so that the liquid supply capability and the atomization efficiency of the atomization core 10 are ensured, and the beauty of the atomizer is improved. It may be understood that the axial length of the support member 11 is selected according to actual needs, for example, the axial length of the support member 11 is determined according to the size of a specific smoke cartridge.

In an embodiment, the hole diameter of the liquid guiding hole 11b ranges from 0.2 mm to 0.5 mm. In this way, to some extent, it can be prevented that the flow of the aerosol-forming medium is affected due to the hole diameter of the liquid guiding hole 11b being too small, that is, it can be prevented that the liquid supply capability of the atomization core 10 is reduced; and it can be prevented that the structural strength of the support member 11 is reduced due to the hole diameter of the liquid guiding hole 11b is too large, thereby reducing the service life of the atomization core 10, and if the hole diameter of the liquid guiding hole 11b is too large, the leakage may occur.

In some embodiments, the plurality of liquid guiding holes 11b are distributed on the tube wall in an array, for example, the plurality of liquid guiding holes 11b are distributed in an array. When the support member 11 is a circular ring, the liquid guiding holes 11b are distributed in the radial direction of the support member 11 in an annular array using the axial center of the support member 11 as a center of a circle. A quantity of the liquid guiding holes 11b may be adaptively selected according to needs. The hole diameter of the liquid guiding hole 11b ranges from 0.2 mm to 0.5 mm. For better connection and uniform liquid supply, the hole diameter of the liquid guiding hole 11b needs to be significantly greater than the effective capillary diameter of the liquid guiding core 12, so that the liquid guiding core 12 can better absorb the liquid from the liquid guiding hole 11b. In addition, the hole diameter of the liquid guiding hole 11b needs to be not greater than 0.5 mm, otherwise, poor uniformity of liquid supply at parts of the liquid guiding core 12 may be caused. Therefore, in an optional solution, the hole diameter of the liquid guiding hole 11b is selected between 0.2 mm to 0.5 mm, and the plurality of liquid guiding holes 11b are uniformly distributed corresponding to the liquid guiding core 12.

In conclusion, in the atomization core provided in the embodiments of this application, the liquid guiding channel 11a in the support member 11 is used for e-liquid supply. Compared with a conventional manner in which an elongated cotton core is used, and the e-liquid is supplied from the two ends of the cotton core, a liquid supply area of the support member 11 is a total area of external openings of all liquid guiding channels 11a on the tube wall. In this way, the liquid supply area of the support member 11 is increased, and a larger amount of the liquid can be atomized on the support member 11, so that atomization power can be increased, and in a case of unchanged temperature, an amount of aerosol generated by the atomization core 10 in a unit time is larger. In addition, the cotton core is arranged between the outer wall surface of the support member 11 and the atomization core 10, preventing the liquid from flowing outward from the liquid guiding channel 11a, and reducing a risk of the leakage. In addition, such a liquid supply manner can further increase a liquid supply flow amount of the support member 11 in a unit time, which is conducive to the liquid guiding hole 11b on the support member 11 maintaining a state soaked with liquid. The liquid in the liquid guiding hole 11b can isolate external air, thereby preventing the air from entering the liquid storage cavity in the atomizer from the liquid guiding hole 11b and forming bubbles, to isolate the liquid from the heating mesh 13, and avoiding a risk of damage of the heating mesh 13 caused by dry heating.

In an embodiment, referring to FIG. 1 to FIG. 3 and FIG. 6, the atomization core 10 includes two connecting rings 14 configured for electrical connection. The two connecting rings 14 are respectively sleeved on the two ends of the support member 11, and are electrically connected to the heating mesh 13. In other words, in the atomization core 10, the connecting rings 14 are respectively sleeved on the two ends of the support member 11, abuts against the heating mesh 13, and are electrically connected to the heating mesh 13, so that the atomization core 10 is electrically connected to the power supply assembly through the two connecting rings 14.

It should be noted that a specific material of the connecting rings 14 is not limited herein. For example, the material of the connecting rings 14 is a conductive metal or alloy such as brass, red copper, or aluminum alloy.

It should be noted that specific arranged positions of the connecting rings 14 are not limited herein, as long as the connecting rings 14 are arranged at the two ends of the support member 11, and are electrically connected to the heating mesh 13. For example, in an embodiment, referring to FIG. 1 to FIG. 3, the two connecting rings 14 are both sleeved on the heating mesh 13. In this way, the connecting rings 14 are sleeved on the heating mesh, so that the heating mesh 13 is firmly fixed on the cotton core and the support member 11, and the heating mesh 13 can be electrically connected to the power supply assembly through the connecting rings 14.

In some other embodiments, the two connecting rings 14 are both sleeved on the support member 11, and the two ends of the heating mesh 13 are respectively connected to the two connecting rings 14.

During assembly, the outer periphery of the support member 11 is wrapped with the cotton core tightly, the heating mesh 13 is arranged on the outer periphery of the cotton core, and the connecting rings 14 are sleeved on the two ends of the heating mesh 13, so that the whole heating element is formed.

In the description of this application, the description of the reference terms such as “in an embodiment”, “in some embodiments”, “in some other embodiments”, “in still some other embodiments”, or “for example” means specific features, structures, materials, or characteristics described with reference to the embodiment or exemplary description are included in at least one embodiment or example of the embodiments of this application. In this application, illustrative description for the above terms do not necessarily indicate the same embodiment or example. Besides, the specific features, structures, materials, or characteristics that are described may be combined in proper manners in any one or more embodiments or examples. Further, without contradicting each other, a person skilled in the art may combine the different embodiments or examples described in this application and features of the different embodiments or examples.

The foregoing descriptions are only preferred embodiments of this application and are not intended to limit this application. For a person skilled in the art, various modifications and changes may be made to this application. Any modification, equivalent replacement, or improvement made within the spirit and principle of this application falls within the protection scope of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An atomization core, comprising:
a support member comprising a liquid guiding channel and a liquid guiding hole, the liquid guiding channel running through two ends of the support member in an axial direction of the support member, the liquid guiding hole running through a side wall of the liquid guiding channel in a radial direction of the support member;
a liquid guiding core sleeved on an outer periphery of the support member, an aerosol-forming medium in the liquid guiding channel being guidable to the liquid guiding core through the liquid guiding hole; and
a heating mesh arranged on an outer periphery of the liquid guiding core, the heating mesh being configured to heat and atomize the aerosol-forming medium.

2. The atomization core of claim 1, wherein the heating mesh comprises a woven structure formed by weaving 4 to 64 heating wires.

3. The atomization core of claim 2, wherein a diameter of each heating wire of the heating wires ranges from 0.01 mm to 0.5 mm.

4. The atomization core of claim 2, wherein a material of the heating wires comprises iron chromium aluminum, nickel chromium, a titanium wire, or a nickel wire.

5. The atomization core of claim 1, wherein the liquid guiding core comprises a cotton core.

6. The atomization core of claim 5, wherein the cotton core comprises 8 to 64 strands of yarn.

7. The atomization core of claim 6, wherein a diameter of each strand of yarn of the strands of yarn ranges from 0.05 mm to 0.2 mm, and/or
wherein a fineness of each strand of yarn of the strands of the yarn ranges from 10 to 60.

8. The atomization core of claim 5, wherein a porosity of the cotton core ranges from 0.45 to 0.99, and
wherein a permeability of the cotton core ranges from 1×10⁻¹¹ m² to 1×10⁻⁹ m².

9. The atomization core of claim 1, wherein a thickness of the liquid guiding core ranges from 0.2 mm to 2 mm, and/or
wherein a length of the support member ranges from 3 mm to 30 mm.

10. The atomization core of claim 1, wherein a hole diameter of the liquid guiding hole is less than an inner diameter of the support member, and is greater than an effective capillary diameter of the liquid guiding core, and/or
wherein a hole diameter of the liquid guiding hole ranges from 0.2 mm to 0.5 mm.

11. The atomization core of claim 1, wherein an inner diameter of the support member ranges from 0.5 mm to 2.0 mm, and/or
wherein a wall thickness of the support member ranges from 0.1 mm to 0.2 mm.

12. The atomization core of claim 1, wherein the atomization core comprises two connecting rings configured for electrical connection, the two connecting rings being respectively sleeved on the two ends of the support member and electrically connected to the heating mesh.

13. The atomization core of claim 12, wherein the two connecting rings are both sleeved on the heating mesh.

14. An atomizer, comprising:
a housing; and
the atomization core of claim 1,
wherein a liquid storage cavity and an aerosol outlet channel are provided in the housing,
wherein the liquid storage cavity is configured to store an aerosol-forming medium,
wherein the aerosol outlet channel is configured for an aerosol to flow out of the atomizer, and
wherein the aerosol-forming medium is flowable into the liquid guiding channel through the liquid storage cavity.

15. An aerosol-forming apparatus, comprising:
a power supply assembly; and
the atomizer of claim 14,
wherein the power supply assembly is electrically connected to the atomizer.
